SEQUENCE LISTING (23 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFVQSIISTLT

SEQ ID NO: 4 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 4 aa, synthetic construct
GSGS

SEQ ID NO: 6 - protein, 153 aa, synthetic construct
MYRMQLLSCIALSLALVTNSAPTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFVQSIISTLT

SEQ ID NO: 7 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISNINVIVLELKGSETTFMCEYADETATIVEFLNRWITFVQSIISTLT

SEQ ID NO: 8 - protein, 153 aa, synthetic construct
MYRMQLLSCIALSLALVTNSAPTSSSTKKTQLQLEHLLLDLQMILNGTNNYKNPKLTEMLTFRFYMPKKATELKHLQCLEEELKPLEEVLNLAQSKNFHLRPRDLISNINVIVLELKGSETTFMCEYADETATIVEFLNRWITFVQSIISTLT

SEQ ID NO: 9 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISDINVIVLELKGSETTFMCEYADETATIVEFLNRWITFVQSIISTLT

SEQ ID NO: 10 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISNINDIVLELKGSETTFMCEYADETATIVEFLNRWITFVQSIISTLT

SEQ ID NO: 11 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISNINAIVLELKGSETTFMCEYADETATIVEFLNRWITFVQSIISTLT

SEQ ID NO: 12 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISNINEIVLELKGSETTFMCEYADETATIVEFLNRWITFVQSIISTLT

SEQ ID NO: 13 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISNINVTVLELKGSETTFMCEYADETATIVEFLNRWITFVQSIISTLT

SEQ ID NO: 14 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISDINDIVLELKGSETTFMCEYADETATIVEFLNRWITFVQSIISTLT

SEQ ID NO: 15 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISDINAIVLELKGSETTFMCEYADETATIVEFLNRWITFVQSIISTLT

SEQ ID NO: 16 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISDINEIVLELKGSETTFMCEYADETATIVEFLNRWITFVQSIISTLT

SEQ ID NO: 17 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISNINDTVLELKGSETTFMCEYADETATIVEFLNRWITFVQSIISTLT

SEQ ID NO: 18 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISNINATVLELKGSETTFMCEYADETATIVEFLNRWITFVQSIISTLT

SEQ ID NO: 19 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISNINETVLELKGSETTFMCEYADETATIVEFLNRWITFVQSIISTLT

SEQ ID NO: 20 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISDINDTVLELKGSETTFMCEYADETATIVEFLNRWITFVQSIISTLT

SEQ ID NO: 21 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISDINATVLELKGSETTFMCEYADETATIVEFLNRWITFVQSIISTLT

SEQ ID NO: 22 - protein, 153 aa, synthetic construct
MYRMQLLSCIALSLALVTNSAPTSSSTKKTQLQLEHLLLDLQMILNGINNYKNPKLTEMLTQQFYMPKEATELKHLQCLEEELKPLEEVLNLAQSKNFHLRPRDLISDINETVLELKGSETTFMCEYADETATIVEFLNRWITFVQSIISTLT

SEQ ID NO: 23 - protein, 153 aa, synthetic construct
MYRMQLLSCIALSLALVTNSAPTSSSTKKTQLQLEHLLLDLQMILNGINNYKNPKLTEMLTQQFYMPKKATELKHLQCLEEELKPLEEVLNLAQSKNFHLRPRDLISNINVIVLELKGSETTFMCEYADETATIVEFLNRWITFVQSIISTLT

(19) United States
(12) Patent Application Publication
(10) Pub. No.: US 2026/0258107 A1
(43) Pub. Date: Sep. 3, 2026

(54) IL2 MUTEINS AND USES THEREOF

(71) Applicants: ELPISCIENCE (SUZHOU) BIOPHARMA, LTD., SUZHOU (CN); ELPISCIENCE BIOPHARMA, LTD., SHANGHAI (CN)

(72) Inventors: Xiang XU, SHANGHAI (CN); Jinfeng ZHAO, SHANGHAI (CN); Zhihao WU, SHANGHAI (CN); Hongtao LU, SHANGHAI (CN)

(73) Assignees: ELPISCIENCE (SUZHOU) BIOPHARMA, LTD., SUZHOU, JS (CN); ELPISCIENCE BIOPHARMA, LTD., SHANGHAI (CN)

(21) Appl. No.: 18/881,695

(22) PCT Filed: Jul. 5, 2023
(86) PCT No.: PCT/CN2023/105943

§ 371 Date: Jan. 6, 2025

(30) Foreign Application Priority Data
Jul. 6, 2022 (WO) PCT/CN2022/104035

(51) Int. Cl. C07K 14/55; A61K 38/00; A61P 35/00; C12N 5/071; C12N 15/85

(52) U.S. Cl. CPC C07K 14/55; A61K 38/00; A61P 35/00; C12N 5/0686; C12N 15/85; C07K 2319/30; C12N 2510/04

(57) ABSTRACT

The present disclosure relates to IL2 muteins (or “IL2 mutants”), an isolated polynucleotide encoding the IL2 mutein, an isolated vector containing the polynucleotide, a host cell containing the isolated polynucleotide or the isolated vector which encoding the IL2 mutein, a fusion protein containing the IL2 mutein, a pharmaceutical composition containing the IL2 mutein/muteins, and uses of such molecules in IL2 immunotherapy.

### CROSS-REFERENCE TO RELATED APPLICATIONS

This application claims the priority to the PCT Application No. PCT/CN2022/104035, filed on Jul. 6, 2022. The content of the prior application is considered as a part of the present disclosure and is incorporated herein in its entirety.

### REFERENCE TO THE SEQUENCE LISTING

The Sequence Listing titled DCF230724WO-Seqlisting.xml, which was created on Jul. 5, 2023 and is 24,064 bytes in size, is hereby incorporated by reference in its entirety.

### TECHNICAL FIELD

The present disclosure relates to an IL2 mutein (or “IL2 mutant”), an isolated vector containing the polynucleotide encoding the IL2 mutein, a host cell containing the isolated polynucleotide or the isolated vector which encoding the IL2 mutein, a fusion protein containing the IL2 mutein, a pharmaceutical composition containing the IL2 mutein/muteins, and uses thereof.

### BACKGROUND

The statements in this section merely provide background information related to the present disclosure and do not necessarily constitute prior art.

Interleukin 2 (IL2), a member of the interleukin 2 cytokine subfamily, is a secreted cytokine produced by activated CD4+ and CD8+T lymphocytes, that is important for the proliferation of T and B lymphocytes. IL2 is also known as T-cell growth factor (TCGF). Human IL2 (UniProt: P60568) is composed of a 20-amino acid N-terminal signal peptide and a 133-amino acid polypeptide chain in the mature protein. IL2 exerts crucial functions during immune homeostasis via its effects on regulatory T (Treg) cells, and the optimizing and fine-tuning of effector lymphocyte responses.

IL2 signals through a heterotrimeric receptor complex composed of distinct α, β, and γ chains (also known as CD25, CD122, and CD132, respectively). IL2Rα (CD25) is not generally thought to directly participate in signal transduction, while once IL2Rα binds to IL2, it could increase the overall affinity of the cytokine for the intermediate affinity receptor (IL2Rβγ heterodimer) from the low nanomolar range into the picomolar range (high affinity receptor, IL2Rαβγ heterotrimer) (Tang et. al, Cytokine: X1 (2019) 100001). T cells that express IL2Rα include regulatory T cells (Treg cells), which are essential for suppressing autoimmune inflammation. IL2Rβ and IL2Rγ express on both cytotoxic effector T cells and Treg cells.

IL2 was approved by the FDA for use in metastatic renal cell carcinoma (1992) and metastatic melanoma (1998) (Tang et. al, Cytokine: X1 (2019) 100001). However, IL2 immunotherapy has not been widely adopted because of its toxic adverse effects when administered at high doses (as needed for antitumor immunotherapy), and its ability to stimulate both cytotoxic effector T cells and Treg cells. Activation of Treg cells is an unwanted effect in anticancer IL2 immunotherapy, as Treg cells can dampen effector T cell responses against tumor antigens (Natalia et. al, Trends Immunol. 2015 December; 36(12):763-777).

Thus, there remains a need for IL2 muteins in IL2 immunotherapy with decreased toxic adverse effects.

### SUMMARY

For the above-mentioned purpose, provided herein is a novel IL2 mutein with reduced, disrupted or abolished binding to IL2Rα. In some embodiments, the IL2 mutein comprises an amino acid sequence comprising the amino acid substitution C125V or C125S as compared to wild type IL2 of SEQ ID NO: 1.

In some embodiments, the IL2 mutein is at least 80%, 85%, 86%, 87%, 88%, 89%, 90%, 91%, 92%, 93%, 94%, 95%, 96%, 97%, 98%, 99% identical to the amino acid sequence set forth in SEQ ID NO: 1, 2 or 3.

In some embodiments, the IL2 muteins comprises one or more amino acid substitutions selected from the group consisting of K8, 128, R38, F42, K43, K49, E61, E62, E68, and L72, as compared to the amino acid sequence set forth in SEQ ID NO: 2 or 3.

In some embodiments, the IL2 mutein comprises one or more amino acid substitutions selected from the group consisting of K8R, I28T, R38D/R38A/R38Q/R38T/R38E/R38N, F42E/F42R/F42A/F42H/F42Q, K43E/K43G/K43S/K43A/K43R/K43P/K43Q, K49E, E61R, E62T/E62G, K64M, E68G/E68V/E68S/E68K/E68A and L72S/L72K, as compared to the amino acid sequence set forth in SEQ ID NO: 2 or 3.

In some embodiments, as compared to the amino acid sequence set forth in SEQ ID NO: 2 or 3, the IL2 mutein comprises one or more amino acid substitutions selected from the group consisting of I28T, R38D/R38A/R38Q/R38T/R38E/R38N, K43E/K43G/K43S/K43A/K43R/K43P/K43Q, E61R and E68G/E68V/E68S/E68K/E68A; or
R38D/R38A/R38Q/R38T/R38E/R38N, F42E/F42R/F42A/F42H/F42Q and K43E/K43G/K43S/K43A/K43R/K43P/K43Q; or
R38D/R38A/R38Q/R38T/R38E/R38N, F42E/F42R/F42A/F42H/F42Q, K43E/K43G/K43S/K43A/K43R/K43P/K43Q, K49E; or
R38D/R38A/R38Q/R38T/R38E/R38N, F42E/F42R/F42A/F42H/F42Q, K43E/K43G/K43S/K43A/K43R/K43P/K43Q and E68G/E68V/E68S/E68K/E68A; or
F42E/F42R/F42A/F42H/F42Q and K43E/K43G/K43S/K43A/K43R/K43P/K43Q; or
K8R, R38D/R38A/R38Q/R38T/R38E/R38N, F42E/F42R/F42A/F42H/F42Q, K43E/K43G/K43S/K43A/K43R/K43P/K43Q, E62T/E62G, E68G/E68V/E68S/E68K/E68A and L72S/L72K; or
R38D/R38A/R38Q/R38T/R38E/R38N, E62T/E62G, E68G/E68V/E68S/E68K/E68A and L72S/L72K; or
R38D/R38A/R38Q/R38T/R38E/R38N, F42E/F42R/F42A/F42H/F42Q, K49E, K43E/K43G/K43S/K43A/K43R/K43P/K43Q and E68G/E68V/E68S/E68K/E68A. K64M,

In some embodiments, as compared to the amino acid sequence set forth in SEQ ID NO: 2 or 3, the IL2 mutein comprises amino acid substitutions
R38D, K43E/K43G and E61R;
R38A, F42E and K43G;
R38E, F42Q and K43Q;
R38A, F42E, K43G, E62T, E68G and L72S/L72K;
R38A, F42E, K43G, and E68G;
R38Q/R38T, F42R/F42A, K43S/K43A, and E68G/E68V;
R38A/R38T/R38E, E62T, E68S and L72K;
E62T/E62G, E68G and L72S;
R38E/R38N, K43R and I28T;
R38E/R38N, K43R, I28T and E68K;
F42H/F42Q and K43Q;
R38E, F42H/F42Q, K43Q and K49E;
R38D/R38E, F42Q, K43G/K43Q and K64M;
R38D/R38E, F42Q, K43G/K43Q and K49E; or
R38A/R38T, F42E/F42A, K43P, E62G, E68A and K8R.

In some embodiments, the IL2 mutein comprises amino acid substitutions
R38D, K43E and E61R;
R38A, F42E and K43G;
R38E, F42Q and K43Q;
R38A, F42E, K43G, E62T, E68G and L72S;
R38A, F42E, K43G and E68G;
R38Q, F42R, K43S and E68V;
R38T, F42A, K43A and E68G;
R38E, E62T, E68S and L72K;
E62T, E68G and L72S;
I28T, R38E and K43R;
I28T, R38N, K43R and E68K;
F42H and K43Q;
R38E, F42Q, K43Q and K49E;
R38D, F42Q, K43G and K64M; or
R38T, F42E, K43P, E62G, E68A and K8R, as compared to the amino acid sequence set forth in SEQ ID NO: 2 or 3.

In some embodiments, the IL2 mutein comprises amino acid substitutions N88D, V91D/V91A/V91E and/or 192T as compared to the amino acid sequence set forth in SEQ ID NO: 2 or 3.

In some embodiments, as compared to the amino acid sequence set forth in SEQ ID NO: 2 or 3, the IL2 mutein comprises amino acid substitutions
R38E, F42Q, K43Q, K49E and N88D;
R38E, F42Q, K43Q, K49E and V91D/V91A/V91E;
R38E, F42Q, K43Q, K49E and I92T;
R38E, F42Q, K43Q, K49E, N88D and V91D/V91A/V91E;
R38E, F42Q, K43Q, K49E, V91D/V91A/V91E and 192T;
R38E, F42Q, K43Q, K49E, N88D, V91D/V91A/V91E and I92T;
I28T, R38E, K43R and N88D;
I28T, R38E, K43R and V91D/V91A/V91E;
I28T, R38E, K43R and I92T;
I28T, R38E, K43R, N88D and V91D/V91A/V91E;
I28T, R38E, K43R, V91D/V91A/V91E and 192T; or
I28T, R38E, K43R, N88D, V91D/V91A/V91E and I92T.

In some embodiments, the IL2 mutein is at least 90%, 95%, 96%, 97%, 98%, 99% identical to the amino acid sequence set forth in any one of SEQ ID NOs: 7-23.

In some embodiments, the IL2 mutein is different from the amino acid sequence set forth in any one of SEQ ID NOs: 7-23 by no more than 10, 9, 8, 7, 6, 5, 4, 3, 2 or 1 amino acid.

In some embodiments, the IL2 mutein comprises the amino acid sequence set forth in any one of SEQ ID NOs: 7-23.

In some embodiments, the IL2 mutein is capable of reducing the affinity binding to IL2Rβγ heterodimer.

In some embodiments, the IL2 mutein is capable of reducing the affinity binding to IL2Rαβγ heterotrimer by reduced or abolished binding to IL2Rα activated by IL2Rαβγ heterotrimer.

In some embodiments, the IL2 mutein is capable of reducing the immune response of regulatory T cells.

In some embodiments, the IL2 mutein binds to IL2Rβγ heterodimer with KD (affinity constant) of more than 0.1E-11M, more than 9.9E-11M, or more than 1E-10M.

In some embodiments, the IL2 mutein binds to IL2Rα with KD (affinity constant) of more than 1.0E-12M.

In some embodiments, the IL2 mutein improves the thermostability with Tm value of not less than 50° C., not less than 55° C., not less than 60° C., or not less than 65° C.

In one aspect, the present disclosure also provides an isolated polynucleotide encoding the IL2 mutein as described herein.

In one aspect, the present disclosure also provides an isolated vector comprising the polynucleotide as described herein.

In one aspect, the present disclosure also provides a host cell comprising the isolated polynucleotide as described herein or the isolated vector as described herein. In some embodiments, the host cell includes HEK293 cells.

In one aspect, the present disclosure also provides a fusion protein comprising the IL2 mutein as described herein. In some embodiments, the IL2 mutein is fused to a Fc region, optionally via a linker.

In one aspect, the present disclosure also provides a pharmaceutical composition comprising the IL2 mutein as described herein, or the isolated polynucleotide as described herein, or the isolated vector as described herein, or the host cell as described herein, or the fusion protein as described herein, and a pharmaceutically acceptable carrier.

In one aspect, the present disclosure also provides the use of the IL2 mutein as described herein, or the isolated polynucleotide as described herein, or the isolated vector as described herein, or the host cell as described herein, or the fusion protein as described herein, or the pharmaceutical composition as described herein in the manufacture of a therapeutic agent for diagnosing, preventing or treating a disease, disorder or condition.

In one aspect, the present disclosure also provides a method of diagnosing, preventing or treating a disease, disorder or condition in a subject in need thereof, comprising administrating to the subject a therapeutically effective amount of IL2 mutein as described herein, or the isolated polynucleotide as described herein, or the isolated vector as described herein, or the host cell as described herein, or the fusion protein as described herein, or the pharmaceutical composition described herein.

In one aspect, present the disclosure also provides a method of enhancing the immune response to a IL2 related disease, disorder or condition in a subject in need thereof, comprising administrating to the subject a therapeutically effective amount of IL2 mutein as described herein, or the isolated polynucleotide as described herein, or the isolated vector as described herein, or the host cell as described herein, or the fusion protein as described herein, or the pharmaceutical composition described herein.

In some embodiments, the disease, disorder or condition comprises a tumor.

In one aspect, the present disclosure also provides a method of reducing IL2 related adverse effects in antitumor immunotherapy in a subject in need thereof, comprising administrating to the subject a therapeutically effective amount of the IL2 mutein, the isolated polynucleotide, the isolated vector, the host cell, the fusion protein or pharmaceutical composition described above.

In some embodiments, the subject is mammals including human.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 shows tetrameric structure of IL2-IL2Rα-IL2Rβ-γc (PDB ID: 2B5I).

FIG. 2 shows the position of C125 residue in IL2 protein (PDB ID: 2B5I). The C125 residue is located in the hydrophobic core of the 4-helix bundle.

FIG. 3 shows the purity analysis of the IL2 mutants with C125 residue substitution. FIG. 3A shows SDS-PAGE (sodium dodecyl sulfate polyacrylamide gel electrophoresis) analysis under reducing (R) and non-reducing (NR) conditions of the purified IL2-WT, IL2-C125S and IL2-C125V. IL2-WT refers to wild type human IL2 protein, IL2-C125S refers to the IL2-WT with C125S substitution, IL2-C125V refers to the IL2-WT with C125V substitution. FIG. 3B shows SEC-HPLC (size exclusion chromatography-high performance liquid chromatography) analysis of the purified IL2-WT, IL2-C125S and IL2-C125V.

FIG. 4 shows the detection by Octet rule of IL2Rα binding avidity of IL2-WT and the IL2 mutants with C125V substitution, nm at vertical ordinate refers to the unit of binding size.

FIG. 5 shows the detection by Octet rule of IL2Rβγ binding avidity of IL2-WT and the IL2 mutants with C125V substitution, nm at vertical ordinate refers to the unit of binding size.

FIG. 6 shows the detection by Octet rule of IL2Rα binding of the IL2 variants ESE01.004, ESE01.006, ESE01.008, ESE01.010 (FIG. 6A), and ESE01.016, ESE01.017, ESE01.018, ESE01.019, ESE01.020, ESE01.042 and IL2-C125V (FIG. 6B).

FIG. 7 shows the detection by Octet rule of IL2Rβγ binding of the IL2 variants ESE01.004, ESE01.006, ESE01.008, ESE01.010, ESE01.016, ESE01.017 (FIG. 7A), and ESE01.018, ESE01.019, ESE01.020, ESE01.042 and IL2-C125V (FIG. 7B).

FIG. 8 shows the affinity detection of IL2Rβγ binding of the IL2 variants. The detection by Octet rule of IL2Rβγ binding of the IL2 variants ESE01.027, ESE01.028, ESE01.029, ESE01.030, ESE01.031, ESE01.032, ESE01.033 and ESE01.034 are showed in FIG. 8A, and ESE01.035, ESE01.036, ESE01.037, ESE01.038, ESE01.039 and ESE01.040 are showed in FIG. 8B. The BLI affinity detection assay of IL2Rβγ binding of the IL2 variants ESE01.027, ESE01.028, ESE01.032, ESE01.033 and ESE01.034 and ESE01.035 are showed in FIG. 8C, and ESE01.036, ESE01.037, ESE01.038, ESE01.039 and ESE01.040 are showed in FIG. 8D.

FIG. 9 shows the binding ability to IL2Rβγ by FACS of the IL2 variants (ESE01.027, ESE01.028, ESE01.029, ESE01.030, ESE01.031, ESE01.032, ESE01.033, ESE01.034, ESE01.035, ESE01.036, ESE01.037, ESE01.038, ESE01.039, ESE01.040 and ESE01.042), ESE01.019, IL2-WT, IL2-C125V and Blank control (unstained control).

### DETAILED DESCRIPTION

The present disclosure is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following description is intended to illustrate some particular embodiments of the invention, and not to exhaustively specify all permutations, combinations and variations thereof.

Unless defined otherwise, all technical and scientific terms used herein have the same meaning as commonly understood by one of ordinary skill in the art to which the disclosure pertains. Although any methods and materials similar or equivalent to those described herein may be used in the practice for testing of the present disclosure, the preferred materials and methods are described herein. In describing and claiming the present disclosure, the following terminology will be used.

The present disclosure provides examples of IL2 (Interleukin 2) muteins with reduced, disrupted or abolished binding to IL2Rα (interleukin 2 receptor subunit alpha). The full amino acid sequence of human IL2 (PDB ID: 2B51) is shown in SEQ ID NO: 1.

IL2 muteins (recombinant human IL2) is an effective antitumor immunotherapy, however, short half-life and severe toxicity limits the optimal dosing of IL2. Just in theory, as shown in FIG. 1, once IL2Rα binds to IL2, IL2 could bind to IL2Rαβγ heterotrimer (high affinity receptor) with greater affinity, which preferentially expands immunosuppressive regulatory T cells (Treg cells) expressing high constitutive levels of IL-2Ra. Regulatory T cells are central to immune system homeostasis and play a major role in maintaining peripheral immune tolerance by dampening (autoreactive) effector T cells. Expansion of Tregs represents an undesirable effect of IL2 for cancer immunotherapy.

IL2 muteins with improved and selective immune stimulatory capacities could promote the expansion and activity of effector T cells while minimizing Treg cells and reduce anti-inflammatory effect.

### Definition

The term “mutated”, “mutation”, “mutein” and “mutant” are interchangeably used herein. Typically, and preferably, said a mutated amino acid or a mutation is an exchange of one amino acid by one or more amino acids, an insertion, a deletion or a combination thereof. Most preferably, said a mutated amino acid or mutation is an exchange of a single amino acid by a different single amino acid.

As used herein, “IL2 mutein” or “IL2 variants” means the muteins derived from the sequence as set forth in SEQ ID NO: 1, be mutated in one or more amino acid, said a mutated amino acid or a mutation is an exchange of one amino acid by one or more amino acids, an insertion, a deletion or a combination thereof. Most preferably, said a mutated amino acid or mutation is an exchange of a single amino acid by a different single amino acid. The IL2 muteins at least contain the sequence shown in any one of SEQ ID NOs: 1-3 and have one or more amino acid exchanged by a different single amino acid.

As used herein, the terms “polynucleotide” refers to polymers of nucleotides of any length of at least two nucleotides, and include, without limitation, DNA, RNA, DNA/RNA hybrids, and modifications thereof.

As used herein, the terms “vector” refers to a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase).

As used herein, the terms “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells.

As used herein, the terms “fusion protein” refers to a protein consisting of at least two domains that are encoded by separate genes that have been joined so that they are transcribed and translated as a single unit, producing a single polypeptide. In some embodiments, fusion proteins can be created in vivo, for example, as the result of a chromosomal rearrangement. In some embodiments, the fusion protein is a Fc-fusion protein. In some embodiments, fusion proteins are constructed by the linking of two protein domains with a peptide linker.

The term “Fc region” as used herein includes native and mutein forms of polypeptides derived from the Fc region of an antibody. Truncated forms of such polypeptides containing the hinge region that promotes dimerization also are included. In some embodiments, the Fc region contains an antibody CH2 and CH3 domain. Along with extended serum half-life, fusion proteins containing Fc moieties (and oligomers formed therefrom) offer the advantage of facile purification by affinity chromatography over Protein A or Protein G columns. Preferred Fc regions are derived from human IgG, which includes IgG1, IgG2, IgG3, and IgG4. One of the functions of the Fc portion of an antibody is to communicate to the immune system when the antibody binds its target.

As used herein, the terms “subject” refers to human and non-human animals. Non-human animals include all vertebrates, such as mice, rats, rabbits, cats, dogs, pig, monkey, chimpanzee, gorilla, and the like. Except when noted, the term “patient” or “subject” are used herein interchangeably.
IL2 refers to Interleukin 2. The term “IL2” and “IL-2” are interchangeably used herein.
IL2Rα represents interleukin 2 receptor subunit alpha, most preferably, represents human interleukin 2 receptor subunit alpha. The term “IL2Rα”, “IL-2Rα” and “CD25” are interchangeably used herein.
IL2Rβ represents interleukin 2 receptor subunit beta, most preferably, represents human interleukin 2 receptor subunit beta. The term “IL2RB”, “IL-2RB” and “CD122” are interchangeably used herein.
IL2Rγ represents interleukin 2 receptor subunit gamma. Most preferably, represents human interleukin 2 receptor subunit gamma, or IL2RG. The term “IL2Rγ”, “IL-2Rγ”, “IL2RG”, “IL-2RG” and “CD132” are interchangeably used herein.
IL2Rβγ refers to the heterodimer of IL2Rβ and IL2Rγ. In some embodiments, IL2Rβγ is the intermediate affinity receptor of IL2. The term “IL2Rβγ” and “IL-2Rβγ” are interchangeably used herein.
IL2RαBy refers to the heterotrimer of IL2Rα, IL2Rβ and IL2Rγ. In some embodiments, IL2RαBγ is the high affinity receptor of IL2. The term “IL2RαBγ” and “IL-2RαBγ” are interchangeably used herein.

### IL2 Muteins

Provided herein is a novel IL2 mutein with reduced, disrupt or abolished binding to IL2Rα, further provided herein is a IL2 mutein with C125 residue substitution.

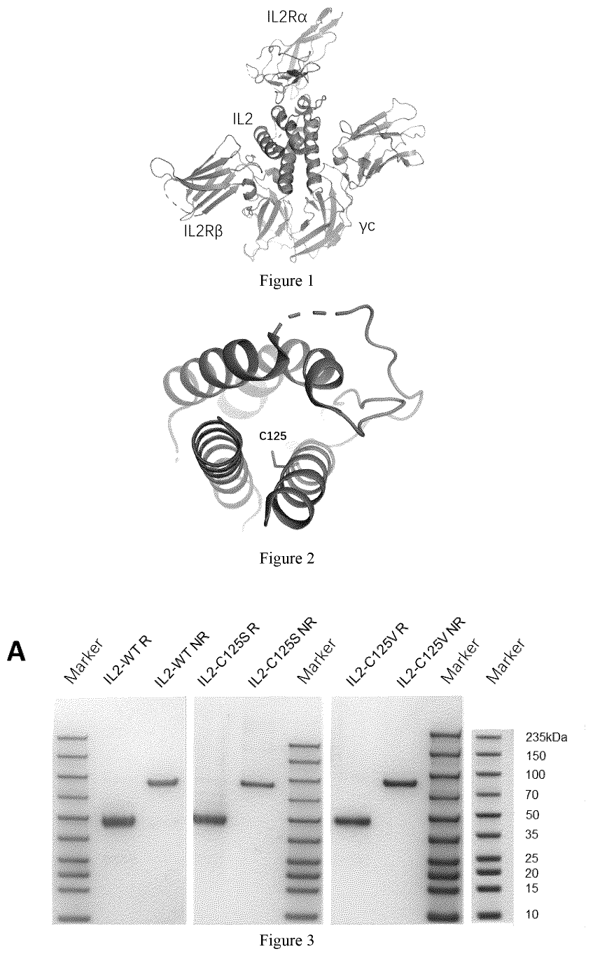

As shown in FIG. 2, the C125 residue is located in the hydrophobic core of the 4-helix bundle. To reduce IL2Rα binding affinity of IL2 mutein, in some embodiments, the IL2 mutein contains an amino acid sequence having the amino acid substitution C125V or C125S as compared to wild type IL2.

In some embodiments, wild type IL2 is human IL2 of SEQ ID NO: 1.

In some preferred embodiments, the IL2 mutein contains an amino acid sequence containing the amino acid substitution C125S as compared to wild type IL2 of SEQ ID NO: 1.

In some embodiments, the IL2 mutein contains an amino acid sequence set forth in SEQ ID NO: 2 and has one or more amino acid substitutions.

In some preferred embodiments, the IL2 mutein contains an amino acid sequence containing the amino acid substitution C125V as compared to wild type IL2 of SEQ ID NO: 1.

Furthermore, in some embodiments, the IL2 mutein contains an amino acid sequence set forth in SEQ ID NO: 3 and has one or more amino acid substitutions.

In some embodiments, the IL2 mutein is at least 80%, 85%, 86%, 87%, 88%, 89%, 90%, 91%, 92%, 93%, 94%, 95%, 96%, 97%, 98%, 99% identical to the amino acid sequence set forth in SEQ ID NO: 1, 2 or 3.

In some embodiments, the IL2 mutein contains one or more amino acid substitutions selected from the group consisting of K8, 128, R38, F42, K43, K49, E61, E62, E68, and L72, as compared to the amino acid sequence set forth in SEQ ID NO: 2 or 3.

In some embodiments, the IL2 mutein is mutated at position K8, 128, R38, F42, K43, K49, E61, E62, K64, E68 and/or L72 compared to wild type IL2.

In some preferred embodiments, the IL2 mutein also contains the following substitutions: K8R, I28T, R38D, R38A, R38Q, R38T, R38E, R38N, F42E, F42R, F42A, F42H, F42Q, K43E, K43G, K43S, K43A, K43R, K43P, K43Q, K49E, E61R, E62T, E62G, K64M, E68G, E68V, E68S, E68K, E68A, L72S, or L72K, or combination thereof, as compared to wild type IL2.

In the present disclosure, the substitution, e.g., K8R, means that, amino acid “K” is substituted by amino acid “R” at position 8. Unless defined otherwise, the abbreviation of amino acid used herein have the same meaning as commonly understood by one of ordinary skill in the art to which the disclosure pertains. The amino acid in the present disclosure is represented as standard single-letter code according to the standard IUPAC (International Union of Pure and Applied Chemistry) amino acid abbreviation.

The term “position” refers to the amino acid residue numbering counting from the N-terminal of a mutein, polypeptide or protein. When there's a signal peptide, the amino acid residue numbering could start from the first amino acid following the signal peptide, or from the first amino acid of the mature protein. Take human IL2 for example, human IL2 is composed of a 20-amino acid N-terminal signal peptide and a 133-amino acid polypeptide chain (the mature protein). The K8R substitution refers to, at the position 8, i.e., the 28th amino acid of human IL2 (SEQ ID NO: 1), the amino acid “K” is substituted by amino acid “R”.

In some embodiments, the “position” is defined by Kabat numbering. The Kabat numbering is well known to those skilled in the art, see, for example, Kabat, E. A. et al. Sequences of Proteins of Immunological Interest, 1991.

In some embodiments, an amino acid could be substituted by amino acid, e.g., R38D/R38A/R38Q/R38T/R38E/R38N, which means the amino acid substitution R38D, R38A, R38Q, R38T, R38E or R38N.

In some embodiments, the IL2 mutein contains one or more amino acid substitutions which could reduce, disrupt or abolish the binding to IL2a, the substitutions are selected from the group consisting of K8R, I28T, R38D/R38A/R38Q/R38T/R38E/R38N, F42E/F42R/F42A/F42H/F42Q, K43E/K43G/K43S/K43A/K43R/K43P/K43Q, K49E, E61R, E62T/E62G, K64M, E68G/E68V/E68S/E68K/E68A, and L72S/L72K, as compared to the amino acid sequence set forth in SEQ ID NO: 2 or 3.

Furthermore, in some embodiments, as compared to the amino acid sequence set forth in SEQ ID NO: 2 or 3, the IL2 mutein contains one or more amino acid substitutions selected from the group consisting of I28T, R38D/R38A/R38Q/R38T/R38E/R38N, K43E/K43G/K43S/K43A/K43R/K43P/K43Q, E61R and E68G/E68V/E68S/E68K/E68A; or
R38D/R38A/R38Q/R38T/R38E/R38N, F42E/F42R/F42A/F42H/F42Q and K43E/K43G/K43S/K43A/K43R/K43P/K43Q; or
R38D/R38A/R38Q/R38T/R38E/R38N, F42E/F42R/F42A/F42H/F42Q, K43E/K43G/K43S/K43A/K43R/K43P/K43Q, K49E; or
R38D/R38A/R38Q/R38T/R38E/R38N, F42E/F42R/F42A/F42H/F42Q, K43E/K43G/K43S/K43A/K43R/K43P/K43Q and E68G/E68V/E68S/E68K/E68A; or
F42E/F42R/F42A/F42H/F42Q and K43E/K43G/K43S/K43A/K43R/K43P/K43Q; or
K8R, R38D/R38A/R38Q/R38T/R38E/R38N, F42E/F42R/F42A/F42H/F42Q, K43E/K43G/K43S/K43A/K43R/K43P/K43Q, E62T/E62G, E68G/E68V/E68S/E68K/E68A and L72S/L72K; or
R38D/R38A/R38Q/R38T/R38E/R38N, E62T/E62G, E68G/E68V/E68S/E68K/E68A and L72S/L72K; or
R38D/R38A/R38Q/R38T/R38E/R38N, F42E/F42R/F42A/F42H/F42Q, K43E/K43G/K43S/K43A/K43R/K43P/K43Q and E68G/E68V/E68S/E68K/E68A. K49E, K64M,

In some embodiments, as compared to the amino acid sequence set forth in SEQ ID NO:2 or 3, the IL2 mutein contains amino acid substitutions
R38D, K43E/K43G and E61R;
R38A, F42E and K43G;
R38E, F42Q and K43Q;
R38A, F42E, K43G, E62T, E68G and L72S/L72K;
R38A, F42E, K43G, and E68G;
R38Q/R38T, F42R/F42A, K43S/K43A, and E68G/E68V;
R38A/R38T/R38E, E62T, E68S and L72K;
E62T/E62G, E68G and L72S;
R38E/R38N, K43R and I28T;
R38E/R38N, K43R, I28T and E68K;
F42H/F42Q and K43Q;
R38E, F42H/F42Q, K43Q and K49E;
R38D/R38E, F42Q, K43G/K43Q and K64M;
R38D/R38E, F42Q, K43G/K43Q and K49E; or
R38A/R38T, F42E/F42A, K43P, E62G, E68A and K8R.

In some embodiments, the IL2 mutein contains amino acid substitutions
R38D, K43E and E61R;
R38A, F42E and K43G;
R38E, F42Q and K43Q;
R38A, F42E, K43G, E62T, E68G and L72S;
R38A, F42E, K43G and E68G;
R38Q, F42R, K43S and E68V;
R38T, F42A, K43A and E68G;
R38E, E62T, E68S and L72K;
E62T, E68G and L72S;
I28T, R38E and K43R;
I28T, R38N, K43R and E68K;
F42H and K43Q;
R38E, F42Q, K43Q and K49E;
R38D, F42Q, K43G and K64M; or
R38T, F42E, K43P, E62G, E68A and K8R, as compared to the amino acid sequence set forth in SEQ ID NO: 2 or 3.

In some embodiments, provided herein is a the IL2 mutein with enhanced binding affinity to IL2Rβγ. In some embodiments, the IL2 mutein contains amino acid substitutions N88D, V91D/V91A/V91E and/or 192T as compared to the amino acid sequence set forth in SEQ ID NO: 2 or 3.

In some embodiments, as compared to the amino acid sequence set forth in SEQ ID NO: 2 or 3, the IL2 mutein contains amino acid substitutions R38E, F42Q, K43Q, K49E and N88D;
R38E, F42Q, K43Q, K49E and V91D/V91A/V91E;
R38E, F42Q, K43Q, K49E and I92T;
R38E, F42Q, K43Q, K49E, N88D and V91D/V91A/V91E;
R38E, F42Q, K43Q, K49E, V91D/V91A/V91E and I92T;
R38E, F42Q, K43Q, K49E, N88D, V91D/V91A/V91E and I92T;
I28T, R38E, K43R and N88D;
I28T, R38E, K43R and V91D/V91A/V91E;
I28T, R38E, K43R and I92T;
I28T, R38E, K43R, N88D and V91D/V91A/V91E;
I28T, R38E, K43R, V91D/V91A/V91E and 192T; or
I28T, R38E, K43R, N88D, V91D/V91A/V91E and I92T.

In some embodiments, the IL2 mutein contains the mutations which can reduce, disrupt or abolish binding to IL2Rα. In some embodiments, the IL2 mutein contains the mutations which can reduce binding to IL2Rβγ.

In some embodiments, the IL2 mutein is having at least 90%, 95%, 96%, 97%, 98%, 99% percent identity with the amino acid sequence set forth in any one of SEQ ID NOs: 7-23.

The method regarding how to determine the identity percentage of two amino acid sequences is known in the art, include but not limited to, BLAST (Basic Local Alignment Search Tool) on NCBI.

In some embodiments, the IL2 mutein is different from the amino acid sequence set forth in any one of SEQ ID NOs: 7-23 by no more than 10, 9, 8, 7, 6, 5, 4, 3, 2 or 1 amino acid.

Furthermore, in some embodiments, the IL2 mutein contains the amino acid sequence set forth in any one of SEQ ID NOs: 7-23.

In some embodiments, the IL2 mutein is derived from the human IL2. In some embodiments, the IL2 mutein is derived from the non-human vertebrates, include but not limited to, mice, rats, rabbits, cats, dogs, pig, monkey, chimpanzee and gorilla.

### IL2 Mutein Characteristics

The IL2 mutein provided herein can reduce, disrupt or abolish the affinity binding to IL2Rα. Meanwhile, the IL2 mutein provided herein can reduce the affinity binding to IL2Rβγ heterodimer.

In some embodiments, the IL2 mutein provided herein can reduce, disrupt or abolish the affinity binding to IL2Rαβγ heterotrimer.

In some embodiments, the IL2 mutein is capable of enhancing the immune response by selectively activating effector T cells and can promote the expansion and activity of effector T cells, for example, by increasing CD8+ effector T cell proliferation.

In some embodiments, the IL2 mutein is capable of reducing the activation of Treg cells, or number of Treg cells. Thus, the IL2 muteins provided herein decrease toxic adverse effect in IL2 immunotherapy.

In some embodiments, the IL2 muteins improves the selective immune stimulatory capacities and reduces anti-inflammatory effect mediated by Treg cells.

In some embodiments, the IL2 muteins with improved and selective immune stimulatory capacities could promote the expansion and activity of effector T cells while minimizing Treg cells and reduce anti-inflammatory effect.

In some embodiments, wherein the IL2 mutein is capable of reducing the affinity binding to IL2Rαβγ heterotrimer by reduced or abolished binding to IL2Rα.

In some embodiments, the IL2 mutein is capable of reducing the immune response of regulatory T cells activated by IL2Rαβγ heterotrimer.

In some embodiments, the IL2 mutein binds to IL2Rβγ heterodimer with affinity constant (KD=koff/kon, or KD=Kd/Ka) of more than 0.1E-11M, more than 9.9E-11M, more than 1E-10M, or more than 9E-10M. Kd/koff is dissociation constant, and Ka/kon is association constant.

In some embodiments, the IL2 mutein binds to IL2Rα with KD of more than 1.0E-12M, more than 1.0E-11M, or more than 1.0E-10M.

In some embodiments, the IL2 mutein improves the thermostability with Tm value of not less than 50° C., not less than 55° C., not less than 60° C., or not less than 65° C. In some embodiments, the IL2 mutein has a Tm greater than 63° C., 64° C., 65° C., 66° C., 67° C., 68° C., 69° C. or 70° C.

In some embodiments, the IL2 mutein is expressed in mammalian cells, include but not limited to, HEK293 cells. In some embodiments, the expression level of IL2 mutein is greater than 70 mg/L, greater than 80 mg/L, greater than 85 mg/L, greater than 100 mg/L, greater than 105 mg/L, greater than 110 mg/L, greater than 115 mg/L, greater than 120 mg/L, or greater than 125 mg/L.

In some embodiments, the monomer percentage of IL2 mutein is greater than 50%, greater than 60%, greater than 65%, greater than 68%, greater than 70%, greater than 71%, or greater than 72%. The detection of monomer percentage can use any one method known in the art, such as the SEC-HPLC.

### Polynucleotides, Vectors, Host Cells and Fusion Proteins

The present disclosure also provides an isolated polynucleotide encoding the IL2 mutein as described above which can reduce, disrupt or abolish the affinity binding to IL2Rα, in some embodiments, IL2 mutein can reduce the affinity binding to IL2Rβγ heterodimer.

The polynucleotide is nucleic acid sequence of DNA, RNA, DNA/RNA hybrids, or modifications thereof. In some embodiments, the polynucleotide is a nucleic acid sequence of DNA. In some embodiments, the polynucleotide is a nucleic acid sequence of RNA. DNA or RNA encoding the IL2 mutein is readily isolated and sequenced using conventional procedures known in the art. The encoding DNA or RNA may also be obtained by synthetic methods.

In one aspect, the present disclosure also provides an isolated vector containing the polynucleotide as described herein. The provided isolated polynucleotide can be inserted into a vector for further cloning (amplification of the DNA) or for expression, using recombinant techniques known in the art.

The vector components generally include, but are not limited to, one or more of the following: a signal sequence, an origin of replication, one or more marker genes, an enhancer element, a promoter (e.g., SV40, CMV, EF-1α), and a transcription termination sequence.

In some embodiments, the vector provided herein includes at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g. SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT. RTM, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus.

In one aspect, the present disclosure also provides a host cell containing the isolated polynucleotide as described herein or the isolated vector as described herein.

Vectors containing the polynucleotide sequence encoding the IL2mutein can be introduced to a host cell for cloning or gene expression. Suitable host cells for cloning or expressing the above-described polynucleotide (nucleic acid sequence of DNA, RNA or DNA/RNA hybrids) in the vectors herein are the prokaryote, yeast, or higher eukaryote cells described above.

In some implementations, eukaryotic microbes such as filamentous fungi or yeast are suitable cloning or expression hosts for IL2 mutein encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12, 424), K. bulgaricus (ATCC 16, 045), K. wickeramii (ATCC 24, 178), K. waltii (ATCC 56, 500), K. drosophilarum (ATCC 36, 906), K. thermotolerans, and K. marxianus; yarrowia (EP 402, 226); Pichia pastoris (EP 183, 070); Candida; Trichoderma reesia (EP 244, 234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of IL2 mutein provided herein are derived from multicellular organisms. However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

In some embodiments, the host cell is a mammalian cultured cell line, such as CHO, BHK, NSO, 293 and their derivatives.

In some embodiments, the host cell includes HEK293 cells.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In one aspect, the present disclosure also provides a fusion protein containing the IL2 mutein as described herein. In some embodiments, the IL2 mutein is fused to a Fc region and/or a linker.

In some embodiments, the Fc region is functional and probably mediates the effector function including ADCC (antibody-dependent cell-mediated cytotoxicity) and/or phagocytosis.

In some embodiments, the Fc regions are derived from human IgG, which includes IgG1, IgG2, IgG3, and IgG4. In some embodiments, the Fc region is Anti-hIgG Fc Capture (AHC).

In some embodiments, the Fc region extend the half-life (t_{1/2}) in vivo and/or in vitro of the IL2 muteins described above or the fusion proteins provided herein.

In some embodiments, the fusion protein contains a linker between the Fc region and the IL2 mutein. Many different linker polypeptides are known in the art and may be used in the context of an IL2 mutein Fc-fusion protein.

In some embodiments, the IL-2 mutein Fc-fusion protein contains one or more copies of a peptide consisting of GSGS (SEQ ID NO: 5) between the Fc region and the IL2 mutein.

In some embodiments, the linkers are glycosylated when expressed in the appropriate cells and such glycosylation may help stabilize the protein in solution and/or when administered in vivo. Thus, in certain embodiments, an IL2 mutein fusion protein contains a glycosylated linker between the Fc region and the IL2 mutein region.

### Pharmaceutical Compositions

Provided herein is a pharmaceutical composition that includes the IL2 mutein, the isolated polynucleotide, the isolated vector, the host cell or the fusion protein described above, and a pharmaceutically acceptable carrier. The pharmaceutical compositions may be formulated in any manner known in the art.

Compositions containing one or more of any of the IL2 mutein, the isolated polynucleotide, the isolated vector, the host cell or the fusion protein described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions are formulated to be compatible with their intended route of administration (e.g., intravenous, intraarterial, intramuscular, intradermal, subcutaneous, or intraperitoneal).

Pharmaceutical acceptable carriers for use in the pharmaceutical compositions disclosed herein may include, for example, a sterile diluent (e.g., sterile water or saline), a fixed oil, polyethylene glycol, glycerin, propylene glycol or other synthetic solvents, antibacterial or antifungal agents, such as benzyl alcohol or methyl parabens, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers.

Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

In some embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In some embodiments, a sterile, lyophilized powder is prepared by dissolving an antibody or antigen-binding fragment as disclosed herein in a suitable solvent. The solvent may contain an excipient which improves the stability of other pharmacological components of the powder or reconstituted solution, prepared from the powder. Excipients that may be used include, but are not limited to, water, dextrose, sorbitol, fructose, corn syrup, xylitol, glycerin, glucose, sucrose or other suitable agents.

Data obtained from cell culture assays and animal studies can be used in formulating an appropriate dosage of any given agent for use in a subject. A therapeutically effective amount of therapeutic agent (i.e., IL2 mutein, the isolated polynucleotide, the isolated vector, the host cell, the fusion protein or pharmaceutical composition) will be an amount that treats the disease in a subject, decreases the severity, frequency, and/or duration of one or more symptoms of a disease in a subject. The effectiveness and dosing can be determined by a health care professional or veterinary professional using methods known in the art, as well as by the observation of one or more symptoms of disease in a subject.

Exemplary dose amounts of therapeutic agent (i.e., IL2 mutein, the isolated polynucleotide, the isolated vector, the host cell, the fusion protein or pharmaceutical composition) per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

### Use in Manufacture of the Therapeutic Agent

The present disclosure also provides the use of the IL2 mutein as described above in the manufacture of a therapeutic agent for diagnosing, preventing or treating a disease, disorder or condition.

The present disclosure also provides the use of the isolated polynucleotide described above in the manufacture of a therapeutic agent for diagnosing, preventing or treating a disease, disorder or condition.

The present disclosure also provides the use of the isolated vector described above in the manufacture of a therapeutic agent for diagnosing, preventing or treating a disease, disorder or condition.

The present disclosure also provides the use of the host cell described above in the manufacture of a therapeutic agent for diagnosing, preventing or treating a disease, disorder or condition.

The present disclosure also provides the use of the fusion protein described above in the manufacture of a therapeutic agent for diagnosing, preventing or treating a disease, disorder or condition.

In some embodiments, the therapeutic agent is a drug or a pharmaceutical substance.

In some embodiments, the disease, disorder or condition is a IL2 related disease. The IL2 muteins provided is with decreased toxic adverse effects in IL2 immunotherapy.

In some embodiments, the disease, disorder or condition includes a tumor, without limitation, the tumor can be hematologic tumor or solid tumor. In some embodiments, the tumor can be malignant or benign. In some embodiments, the disease, disorder or condition is a cancer. In some embodiments, at least a cell of the tumor express IL2.

In some embodiments, the therapeutic agent is used to diagnose, prevent or treat a disease, disorder or condition in mammals, such as human. In some embodiments, the therapeutic agent is used to diagnose, prevent or treat a disease, disorder or condition in non-human vertebrates including mice, rats, rabbits, cats, dogs, pig, monkey, chimpanzee or gorilla.

### Methods of Treatment

The above-described IL2 mutein, the isolated polynucleotide, the isolated vector, the host cell or the fusion protein can be used for various therapeutic purposes.

In one aspect, the present disclosure also provides a method of diagnosing, preventing or treating a disease, disorder or condition in a subject in need thereof, including administrating to the subject a therapeutically effective amount of the IL2 mutein, the isolated polynucleotide, the isolated vector, the host cell, the fusion protein or pharmaceutical composition described above.

In one aspect, present the disclosure also provides a method of enhancing the immune response to a IL2 related disease in a subject in need thereof, including administrating to the subject a therapeutically effective amount of the IL2 mutein, the isolated polynucleotide, the isolated vector, the host cell, the fusion protein or the pharmaceutical composition described above.

In some embodiments, the disease, disorder or condition includes a tumor. In some embodiments, the disease, disorder or condition includes a cancer. Without limitation, the methods of treatment reduce the rate of the increase of volume of a tumor in a subject over time, reduce the risk of developing a metastasis, or reduce the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the present disclosure also provides a method of reducing IL2 related adverse effects in antitumor immunotherapy in a subject in need thereof, including administrating to the subject a therapeutically effective amount of the IL2 mutein, the isolated polynucleotide, the isolated vector, the host cell, the fusion protein as described herein.

In some embodiments, the IL2 related adverse effects are caused by Treg cells activating by IL2, IL2-like or the therapeutic agent (e.g., IL2 mutein).

In some embodiments, the subject in need thereof is identified or diagnosed as having a cancer, such as breast cancer, carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma.

As used herein, by an “effective amount” is meant an amount or dosage sufficient to effect beneficial or desired results including halting, slowing, retarding, or inhibiting progression of a disease. An effective amount will vary depending upon, e.g., an age and a body weight of a subject to which the IL2 mutein, polynucleotide encoding IL2 mutein, vector containing the polynucleotide, fusion protein and/or pharmaceutical compositions is to be administered, a severity of symptoms and a route of administration, and thus administration can be determined on an individual basis. An effective amount can be administered in one or more administrations.

A typical daily dosage of an effective amount of therapeutic agent (e.g., IL2mutein or fusion protein) is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the IL2 mutein, the isolated polynucleotide, the isolated vector, the host cell, the fusion protein or pharmaceutical composition described above and, optionally, at least one another therapeutic agent (pharmacologically active substances) be administered to a subject. The subject includes mammals, in some embodiments, the subject includes human. In some embodiments, the subject is human.

### Examples

The invention is further described in the following examples, which do not limit the scope of the invention described in the claims.

### Example 1: General Method

### Gene Synthesis

All the IL2 variants used in examples were fused to the N terminus of human IgG1-LALA (huIgG1 Fc-LALA, SEQ ID NO: 4) via a short linker GSGS (SEQ ID NO: 5).

(SEQ ID NO: 4)
DKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK.

Desired gene segments were synthesized from synthetic oligonucleotides and cloned into pcDNA3.4 vectors. The plasmid DNA was purified from transformed bacteria and concentration was determined by UV spectroscopy. The DNA sequence of the gene fragments was confirmed by DNA sequencing.

### Protein Production

HEK293 cells growing in suspension with a scale of 30 ml were transfected by electroporation with the respective expression vectors. Conditioned medium was collected by centrifugation 4 days after transfection. The IL2 variants were purified by one affinity step using protein A. Resin bound proteins were eluted by 0.05M citric buffer (pH 3.4) and neutralized immediately by Tris buffer (pH 9.0). The purified proteins were dialyzed against phosphate-buffered saline. Concentrations of proteins were determined by UV spectroscopy (Ultraviolet spectroscopy). Protein purity was evaluated by SDS-PAGE and SEC-HPLC (Agilent).

### Protein Thermostability Assessment

Thermostability of the purified variants were assessed using thermal shift assay. The proteins were mixed with freshly diluted Protein Thermal Shift Dye (Thermo Fisher Scientific). The mixtures were then transferred into a 384-well plate and loaded onto the QuantStudio Real-Time PCR system (Thermo Fisher Scientific) for Tm measurement. Temperature was scanned from 25° C. to 99° C. with a ramp rate of 1.6° C./see and a 2-min hold time. The melting curves obtained were analyzed with the Protein Thermal Shift Software (Thermo Fisher Scientific) to obtain the Tm values.

### Measurement of IL2R Binding by IL2 Variants

The affinity of the IL2 variants to the IL-2Rβγheterodimer and the IL-2Rα-subunit was determined by Bio-Layer Interferometry (BLI) technology (Octet Red 96 system). Briefly, the IL2 variants with a concentration of 50 nM were loaded and immobilized on an AHC (Anti-hIgG Fc Capture) sensor until the signal reached 0.6 nm. The sensors were immersed in buffer until the signal returned to baseline. Subsequently, either the HIS-tagged human IL-2R a-subunit or the HIS-tagged human IL-2Rβγheterodimer was applied to the sensors as analytes at 30° C. in kinetics buffer (PALL, USA) in concentrations ranging from 100 nM down to 3.125 nM (1:2 dilution). The following conditions were applied: 90 s for association 300 s for dissociation and 6×5 s for regeneration with regeneration solution (10 mM Glycine, pH 1.5). The curves obtained were fitted with a 1:1 binding model.

### Flow Cytometry (FCM)

IL2Rβγ-overexpressing Raji cells suspended in FACS buffer (buffered saline solution containing fetal bovine serum and 0.09% sodium azide) were stained with serial dilutions (3-fold dilution starting from 1000 nM) of IL2 variants for 1 h on ice and followed by three washes. Subsequently, the cells were incubated with a secondary antibody Alexa Fluor 647-Goat Anti-Human IgG Fc (SouthernBiotech, Cat. No.: 2048-31) for 30 min, followed by three washes. Flow cytometry was performed on a FACSCELESTA (BD Biosciences), and MFI was calculated accordingly.

### Example 2: IL2 Mutation Library Construction and Screening

The overall structure of IL2-IL2Rα-IL2Rβ-γc tetrameric structure (PDB ID: 2B51) is presented in FIG. 1. Analysis of the interface between IL-2 and IL-2Rαrevealed that several residues on IL-2 are involved in the electrostatic and hydrophobic interactions with IL-2Rα. Six of them (R38, F42, K43, E62, E68, L72) were selected to be mutated to disrupt interactions with IL-2Rα using a library approach.

Primers were designed to randomize residues at these selected positions and the site-directed mutagenesis library was constructed by overlapping polymerase chain reaction (PCR) using these primers. The PCR product was transformed into yeast along with linearized pYD1 vector. Yeast cells from the IL-2 library were labeled with human IgG1-Fc-tagged soluble IL-2Rαand biotinylated soluble IL-2Rβγ. The cells were washed, labeled with streptavidin conjugated with R-phycoerythrin (PE) and a mouse anti-human IgG Fc antibody conjugated with FITC. The cells were then sorted on a S3e Cell Sorter (Bio-Rad) to isolate clones with abolished binding to soluble IL-2Rαand comparable binding to soluble IL-2Rβγ, relative to human wild-type IL-2. Three rounds of sorting by flow cytometer were carried out, with expansion of sorted library and induction of surface expression between each sort. DNA from the sorted clones was extracted and mixed with DNA fragment of wildtype IL2 as template for random mutagenesis by error-prone polymerase chain reaction (PCR). The error rate was controlled by using different amounts of templates in the PCR system. The PCR product obtained was further amplified by PCR for adding homology arms. The final PCR product was transformed into yeast along with linearized pYD1 vector. Yeast cells from the new IL2 library were labeled as described above. The cells were then sorted on a S3e Cell sorter (Bio-Rad).

To isolate clones with abolished binding to soluble IL-2Rαand decreased binding to soluble IL-2Rβγ, relative to human wild-type IL-2. Four rounds of sorting by flow cytometry were carried out, with expansion of sorted library and induction of surface expression between each sort. After the fourth sort, sorted pool was plated for colony picking. Picked clones were screened by FACS to confirm binding to IL-2Rαand IL-2Rβγ. Finally, total 14 individual clones were lysed as template and DNA was amplified by PCR. Sequences of the IL2 mutants were determined by DNA sequencing.

### Example 3: Generation and Evaluation of IL2 Variants with C125 Substitution

Sequence analysis showed that there's one unpaired cysteine residue at position 125 of human IL2 (Uniprot: P60568). This residue has been mutated to serine prior to further engineering in available literature (Weiger et al., Eur J Biochem 180, 295-300 (1989), PN4518584). However, based on structure analysis of IL2 (PDB ID: 2B51), the C125 residue is located in the hydrophobic core of the 4-helix bundle (FIG. 1). It was hypothesized that substitution of C125 with hydrophobic residues with small side chain, e.g., alanine (Ala, A), valine (Val, V), leucine (Leu, L) and isoleucine (Ile, I), would improve protein stability.

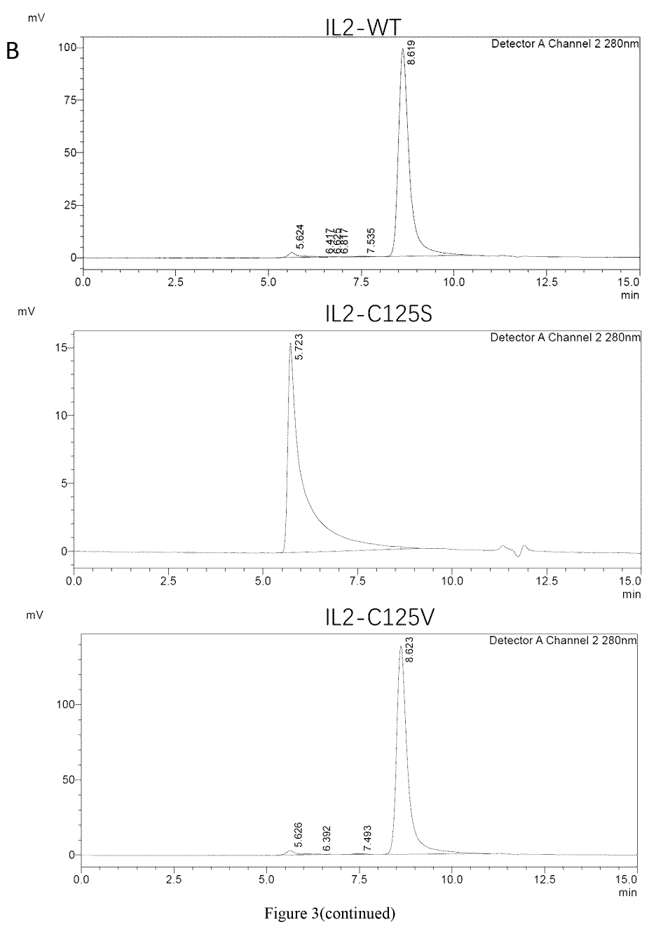

Thus, human wildtype IL2 (IL2-WT, SEQ ID NO: 1), IL2-C125S (SEQ ID NO: 2) and IL2-C125V (SEQ ID NO: 3) were individually fused to Fc tag (human IgG1-LALA), expressed and evaluated for multiple properties. Consistent with hypothesis, SDS-PAGE (FIG. 3A) and SEC-HPLC (FIG. 3B) results showed that the IL2-C125S mutant, which disrupts the hydrophobic core of the 4-helix bundle with hydrophilic serine residue substitution, tended to form soluble aggregates, whereas both wildtype IL2 and the IL2-C125V variant existed mainly as monomers (FIG. 3A, 3B).

In addition, the IL2-C125V variant exhibited much higher expression level as compared to wildtype IL2 (IL2-WT) and the IL2-C125S variant (Table 1). Moreover, IL2-C125V variant also showed higher Tm1 in thermostability test (Table 1), which suggested that the C125V substitution indeed improved stability of IL2 protein.

TABLE 1
Summary of properties of the IL2 mutants
with C125 residue substitution

| | Expression level | Monomer | Tm1 |
|---|---|---|---|
| Variant | (mg/L) | (SEC-280 nm, %) | (° C.) |
| IL2-WT | 139.5 | 97.081 | 58.1 |
| IL2-C125S | 66.7 | Soluble aggregates | N/A |
| IL2-C125V | 192.4 | 97.674 | 59.5 |

* N/A stands for Not Applicable.

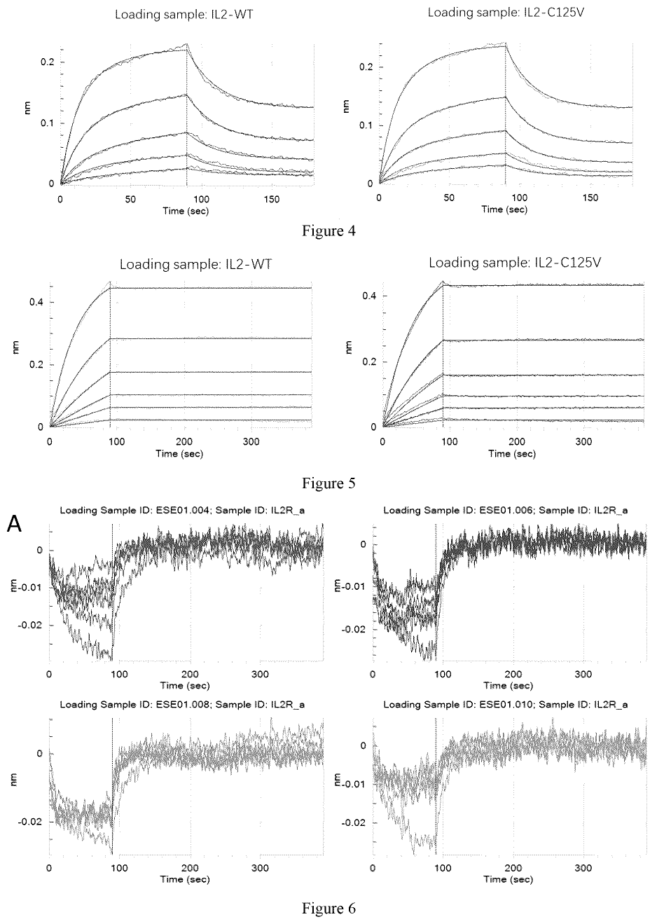

More importantly, compared to wildtype IL2, the C125V substitution did not have negative impacts on binding to IL2Rα (FIG. 4, Table 2) or IL2Rβγ (FIG. 5, Table 3).

TABLE 2
IL2Rα binding avidity of the IL2
mutants with C125 residue substitution

| Variant | KD (M) |
|---|---|
| IL2-C125V | 3.65E−08 |
| IL2-WT | 3.36E−08 |

TABLE 3
IL2Rβγ binding avidity of the IL2
mutants with C125 residue substitution

| Variant | KD (M) |
|---|---|
| IL2-C125V | <1.0E−12 |
| IL2-WT | <1.0E−12 |

Based on these data, C125V substitution of IL2 was used as a default template for further engineering.

### Example 4: Generation and Evaluation of IL2 Variants with Disrupted IL2Rα Binding

After having the sequences of sorted clones with disrupted IL2Rα binding and altered IL2Rβγbinding, further engineering and verification were divided into two steps. The first step aimed to identify the IL2 variant with abolished IL2Rα binding and desired developability; this variant would then be used as a template for the second step, which introduced substitutions of residues involved in IL2Rβγ binding to generate a series of mutations with varying degrees of IL2Rβγ binding.

Hence, in order to identify the IL2 variant with abolished IL2Rα binding, sequences of the selected 14 clones from yeast display library were further analyzed in the IL2-IL2Rα-IL2Rβ-γc tetramer structure. Some of the sequences contained residue substitutions that would impact interactions with IL2Rβγ binding. These residues were back mutated in this step and investigated in the second step. Therefore, sequences of the new 14 IL2 variants (IL2Rα non-binders) were generated (Table 4-1).

TABLE 4-1
Mutations of the potential IL2Rα non-binders

| | Position | | | | | | | | | | | |
|---|---|---|---|---|---|---|---|---|---|---|---|---|
| Library No. | 8 | 10 | 28 | 38 | 42 | 43 | 49 | 61 | 62 | 64 | 68 | 72 |
| IL2-C125V | K | T | I | R | F | K | K | E | E | K | E | L |
| ESE01.004 | | | | D | | E | | R | | | | |
| ESE01.005 | | | | A | E | G | | | T | | G | S |
| ESE01.006 | | | | Q | R | S | | | | | V | |
| ESE01.007 | | | | T | A | A | | | | | G | |
| ESE01.008 | | | | E | | | | | T | | S | K |
| ESE01.010 | | | | A | E | G | | | | | | |
| ESE01.011 | | | | A | E | G | | | | | G | |
| ESE01.012 | | | | | | | | | T | | G | S |
| ESE01.015 | | T | T | N | | R | | | | | K | |
| ESE01.016 | R | | | T | E | P | | | G | | A | |
| ESE01.017 | | | | | H | Q | | | | | | |
| ESE01.018 | | | | D | Q | G | | | | M | | |
| ESE01.019 | | | | E | Q | Q | E | | | | | |
| ESE01.020 | | | T | E | | R | | | | | | |

*The positions are defined with reference to SEQ ID NO: 1. The sequence of Library No. IL2-C125V is set forth in SEQ ID NO: 3.

These IL2 variants with disrupted IL2Rα binding were expressed in HEK293 cells and eventually 9 of them showed various levels of expression and monomer percentage (Table 4-2). The reported IL2-3X variant (SEQ ID NO: 6) with abolished IL2Rα binding but normal IL2Rβγ binding was generated as a reference.

TABLE 4-2
Summary of properties of the potential IL2Rα non-binders

| | Expression level | Monomer | Tm1 |
|---|---|---|---|
| Variant | (mg/L) | (SEC-280 nm, %) | (° C.) |
| IL2-3X | 284.7 | 71.239 | 63.5 |
| (ESE01.004) | | | |
| ESE01.006 | 1 | 14.410 | N/A |
| ESE01.008 | 13.7 | 59.670 | N/A |
| ESE01.010 | 1.7 | 51.019 | N/A |
| ESE01.016 | 49.7 | 52.184 | N/A |
| ESE01.017 | 260.8 | 63.558 | N/A |
| ESE01.018 | 8.7 | 47.289 | N/A |
| ESE01.019 | 251.0 | 94.135 | 65.2 |
| ESE01.020 | 167.3 | 83.673 | 65.5 |
| ESE01.042 | 227.7 | 95.590 | N/A |

*All the variants contain C125V mutation.

Compared to IL2-3X, the variants ESE01.019 (SEQ ID NO: 7) and ESE01.020 (SEQ ID NO: 8) showed higher monomer percentage in SEC-HPLC and higher thermostability in thermal shift assay (Table 4-2). ESE01.019 also exhibited high expression level that reached 251 mg purified protein from 1 liter medium (Table 4-2).

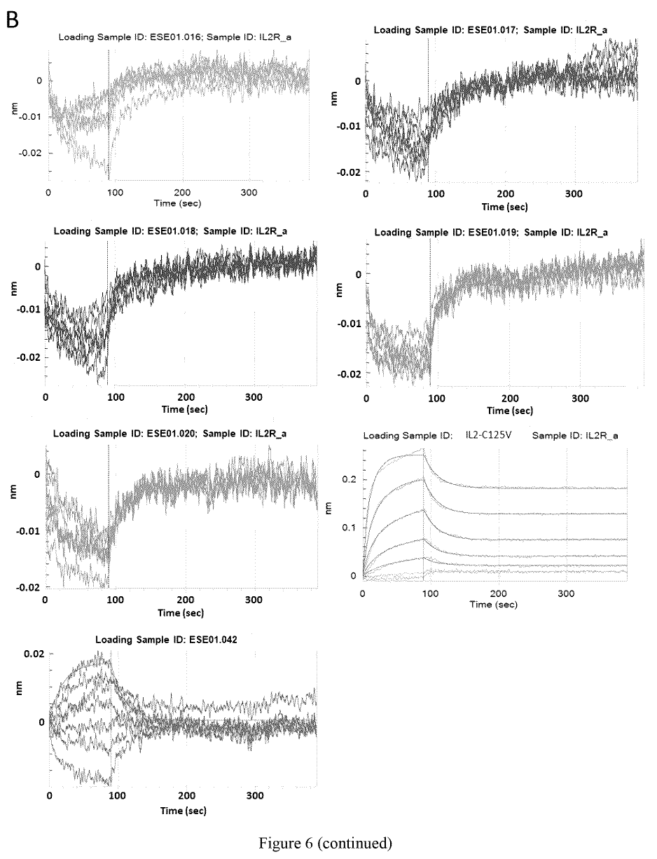

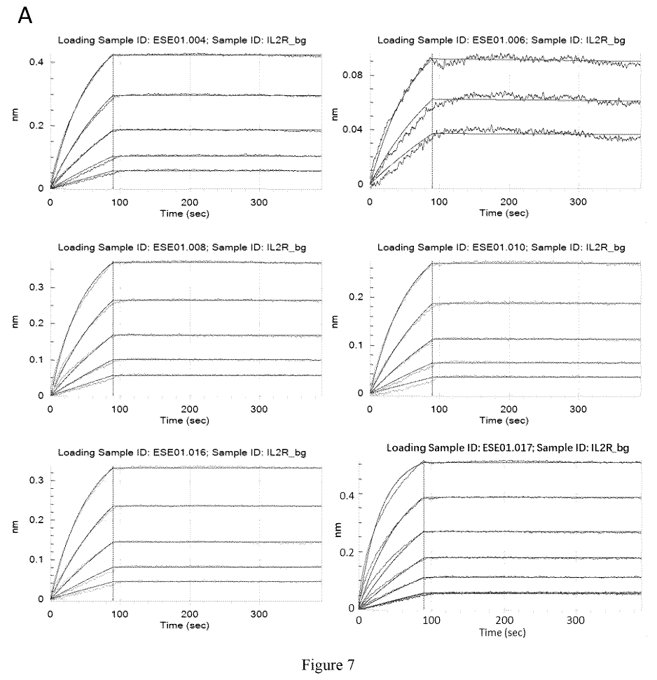

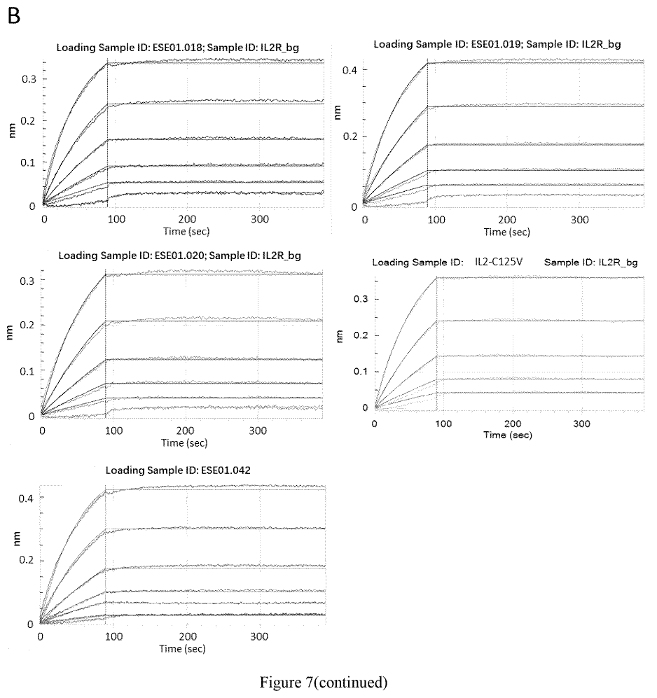

All the 9 variants, as well as IL2-3X, showed abolished IL2Rα binding ability in avidity detection test (FIG. 6A and FIG. 6B). ESE01.019 also showed equal IL2Rβγ binding ability compared to IL2-C125V and IL2-3X (FIG. 7A and FIG. 7B, Table 5).

According to structural analysis of IL2-IL2Rα interface, K49 residue of IL2 which was substituted with a glutamic acid residue in ESE01.019 didn't participate in direct interaction with IL2Rα. Thus, a new variant ESE01.042 that derived from ESE01.019 with back-mutated K49 residue was generated for characterization. Indeed, the ESE01.042 variant exhibited abolished IL2Rαbinding ability in avidity detection test (FIG. 6A and FIG. 6B) and equal IL2Rβγ binding ability compared to IL2-C125V, IL2-3X and ESE01.019 (FIG. 7A and FIG. 7B, Table 5). ESE01.042 also showed comparable expression level and monomer percentage in SEC-HPLC analysis with ESE01.019 (Table 4-2).

TABLE 5
IL2Rβγ binding avidity of the IL2 variants

| Variant | KD (M) |
|---|---|
| IL2-3X | 3.17E−11 |
| (ESE01.004) | |
| ESE01.006 | 4.40E−10 |
| ESE01.008 | 2.83E−11 |
| ESE01.010 | <1.0E−12 |
| ESE01.016 | <1.0E−12 |
| ESE01.017 | 1.56E−11 |
| ESE01.018 | <1.0E−12 |
| ESE01.019 | <1.0E−12 |
| ESE01.020 | <1.0E−12 |
| ESE01.042 | <1.0E−12 |
| IL2-C125V | <1.0E−12 |

### Example 5: Generation and Evaluation of IL2 Variants with Abolished IL2Rα Binding and Reduced IL2Rβγ Binding

Since the ESE01.019 variant showed desired functional properties—no detectable IL2Rαbinding and comparable IL2Rβγ binding—and good protein properties, it was selected as a template for further engineering for IL2Rβγ binding manipulation. Substitutions of three residues, N88D, V91D/A/E and 192T, were identified in library screening mentioned above and combined with each other for introduction into the sequence of the ESE01.019 variant. In total a new series of 14 IL2 variants were designed, expressed and purified in one-step by protein A for assessment of protein properties and IL2 receptor binding. Sequences of new series IL2 variants were shown in Table 6.

TABLE 6
Mutations of the IL2 variants

| | Position | | | | | | |
|---|---|---|---|---|---|---|---|
| Library No. | 38 | 42 | 43 | 49 | 88 | 91 | 92 |
| IL2-WT (wild type) | R | F | K | K | N | V | I |
| ESE01.027 | E | Q | Q | E | D | | |
| ESE01.028 | E | Q | Q | E | | D | |
| ESE01.029 | E | Q | Q | E | | A | |
| ESE01.030 | E | Q | Q | E | | E | |
| ESE01.031 | E | Q | Q | E | | | T |
| ESE01.032 | E | Q | Q | E | D | D | |
| ESE01.033 | E | Q | Q | E | D | A | |
| ESE01.034 | E | Q | Q | E | D | E | |
| ESE01.035 | E | Q | Q | E | | D | T |
| ESE01.036 | E | Q | Q | E | | A | T |
| ESE01.037 | E | Q | Q | E | | E | T |
| ESE01.038 | E | Q | Q | E | D | D | T |
| ESE01.039 | E | Q | Q | E | D | A | T |
| ESE01.040 | E | Q | Q | E | D | E | T |

*The positions are defined with reference to SEQ ID NO: 1.

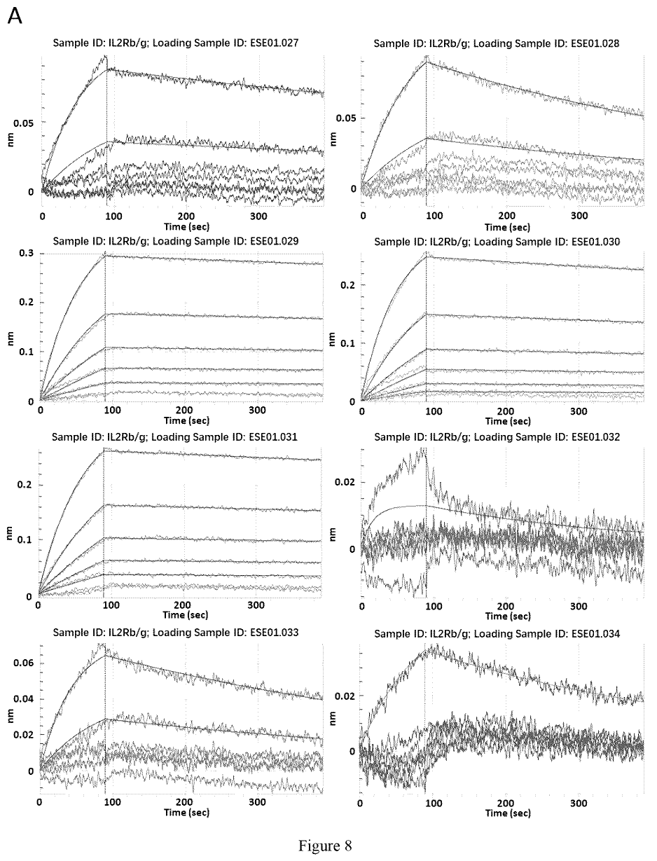

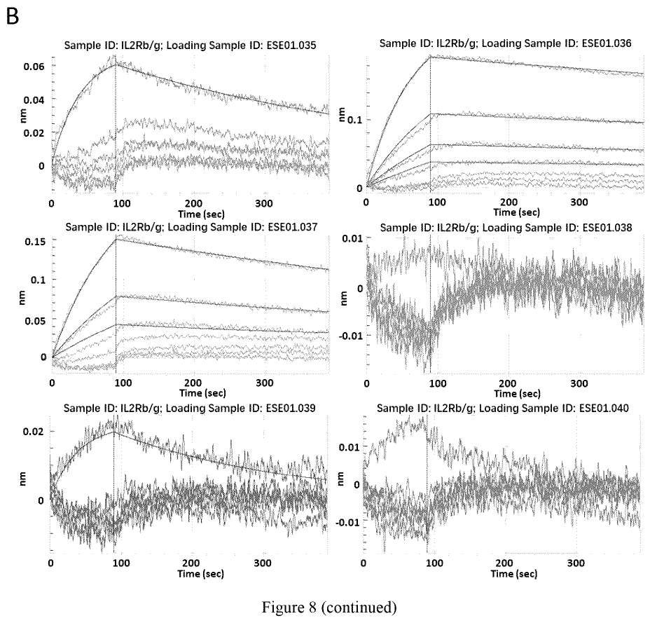

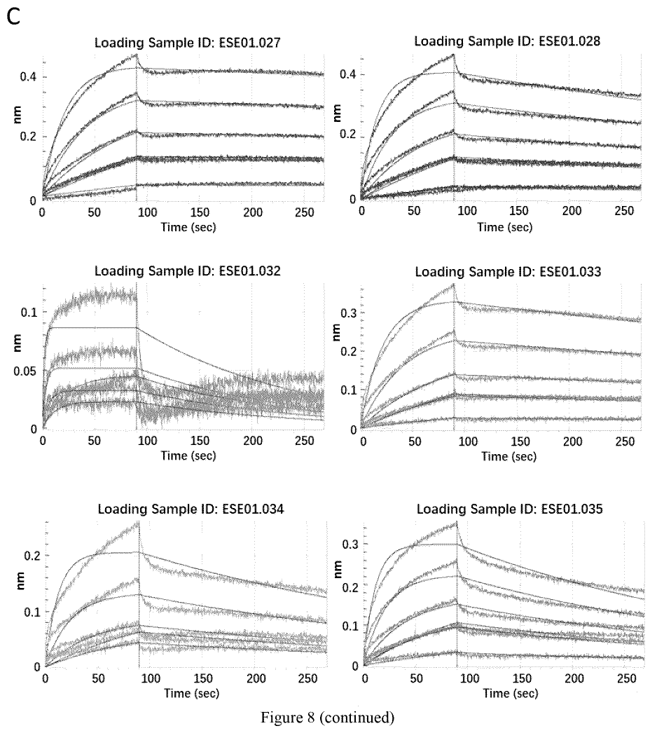

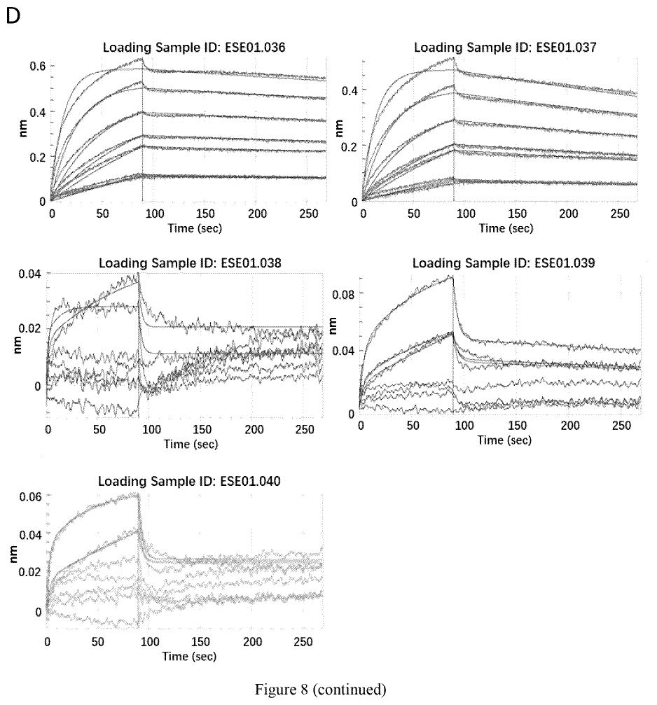

The new series of IL2 variants showed varying levels of IL2Rβγ binding ability (FIG. 8A and FIG. 8B). To further measure IL2Rβγ binding ability of the IL2 variants that showed significantly decreased binding ability, maximum concentration of the HIS-tagged human IL-2Rβγheterodimer was increased from 100 nM to 1000 nM in the BLI affinity detection assay (FIG. 8C and FIG. 8D). Avidity of the series of IL2 variants was listed in Table 7-1.

Yields of these variants were higher than 130 mg/L except for ESE01.030. Molecular purity of these variants, as evaluated by monomer percentage in SEC-HPLC, varied from 79.8% to 97.8%. Tm measurement with DSF suggested all of the new variants had good thermostability with Tm1 higher than 67° C. (Table 7-2).

TABLE 7-1
IL2Rβγ binding avidity of the IL2 variants

| Variant | KD (M) |
|---|---|
| ESE01.027 | 7.02E−09 |
| ESE01.028 | 2.11E−08 |
| ESE01.029 | 1.03E−09 |
| ESE01.030 | 1.51E−09 |
| ESE01.031 | 1.26E−09 |
| ESE01.032 | Undetectable |
| ESE01.033 | 2.06E−08 |
| ESE01.034 | 3.56E−08 |
| ESE01.035 | 3.90E−08 |
| ESE01.036 | 6.50E−09 |
| ESE01.037 | 1.73E−08 |
| ESE01.038 | Undetectable |
| ESE01.039 | 6.60E−06 |
| ESE01.040 | Undetectable |

TABLE 7-2
Summary of properties of the IL2 variants

| | Expression level | Monomer | Tm1 |
|---|---|---|---|
| Variant | (mg/L) | (SEC-280 nm, %) | (° C.) |
| ESE01.027 | 178.3 | 79.847 | 68.2 |
| ESE01.028 | 179.0 | 93.002 | 68.9 |
| ESE01.029 | 182.3 | 83.452 | 69.4 |
| ESE01.030 | 92.3 | 97.770 | 69.4 |
| ESE01.031 | 167.0 | 91.353 | 68.7 |
| ESE01.032 | 153.3 | 95.483 | 69.7 |
| ESE01.033 | 149.0 | 85.951 | 69.4 |
| ESE01.034 | 194.0 | 94.026 | 70.0 |
| ESE01.035 | 159.0 | 88.628 | 67.1 |
| ESE01.036 | 183.3 | 88.818 | 69.7 |
| ESE01.037 | 161.7 | 94.154 | 68.8 |
| ESE01.038 | 172.0 | 88.929 | 69.4 |
| ESE01.039 | 132.0 | 86.322 | 70.2 |
| ESE01.040 | 160.0 | 86.164 | 70.3 |

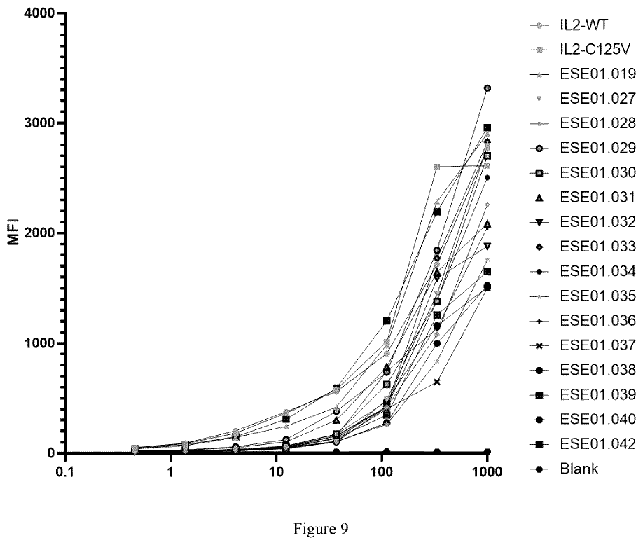

All the 15 variants (ESE01.027, ESE01.028, ESE01.029, ESE01.030, ESE01.031, ESE01.032, ESE01.033, ESE01.034, ESE01.035, ESE01.036, ESE01.037, ESE01.038, ESE01.039, ESE01.040 and ESE01.042) together with IL2-WT, IL2-C125V and ESE01.019 were compared for binding ability to IL2Rβγ by FACS using receptor-overexpressing Raji cells (FIG. 9). Consistently, compared to IL2-WT (SEQ ID NO: 1) and ESE01.019 binding to cell surface IL2Rβγ was dialed down to varying degree among this series of IL2 variants, as expected.

Amino acid sequences mentioned above in the present disclosure are shown in Table 8.

TABLE 8
Amino acid Sequence

| Sequence ID | | Amino acid Sequence |
|---|---|---|
| No. | Name | (Single-letter code) |
| SEQ ID NO: 1 | IL2-WT | MYRMQLLSCIALSLALVTNSAPTSSSTKKTQLQLEHLLLD |
| | (human) | LQMILNGINNYKNPKLTRMLTFKFYMPKKATELKHLQCL |
| | | EEELKPLEEVLNLAQSKNFHLRPRDLISNINVIVLELKGSE |
| | | TTFMCEYADETATIVEFLNRWITFCQSIISTLT |
| SEQ ID NO: 2 | IL2-C125S | MYRMQLLSCIALSLALVTNSAPTSSSTKKTQLQLEHLLLD |
| | | LQMILNGINNYKNPKLTRMLTFKFYMPKKATELKHLQCL |
| | | EEELKPLEEVLNLAQSKNFHLRPRDLISNINVIVLELKGSE |
| | | TTFMCEYADETATIVEFLNRWITFSQSIISTLT |
| SEQ ID NO: 3 | IL2-C125V | MYRMQLLSCIALSLALVTNSAPTSSSTKKTQLQLEHLLLD |
| | | LQMILNGINNYKNPKLTRMLTFKFYMPKKATELKHLQCL |
| | | EEELKPLEEVLNLAQSKNFHLRPRDLISNINVIVLELKGSE |
| | | TTFMCEYADETATIVEFLNRWITFVQSIISTLT |
| SEQ ID NO: 6 | IL2-3X | MYRMQLLSCIALSLALVTNSAPTSSSTKKTQLQLEHLLLD |
| | (ESE01.004) | LQMILNGINNYKNPKLTDMLTFEFYMPKKATELKHLQCL |
| | | ERELKPLEEVLNLAQSKNFHLRPRDLISNINVIVLELKGSE |
| | | TTFMCEYADETATIVEFLNRWITFVQSIISTLT |
| SEQ ID NO: 7 | ESE01.019 | MYRMQLLSCIALSLALVTNSAPTSSSTKKTQLQLEHLLLD |
| | | LQMILNGINNYKNPKLTEMLTQQFYMPKEATELKHLQCL |
| | | EEELKPLEEVLNLAQSKNFHLRPRDLISNINVIVLELKGSE |
| | | TTFMCEYADETATIVEFLNRWITFVQSIISTLT |
| SEQ ID NO: 8 | ESE01.020 | MYRMQLLSCIALSLALVTNSAPTSSSTKKTQLQLEHLLLD |
| | | LQMILNGTNNYKNPKLTEMLTFRFYMPKKATELKHLQCL |
| | | EEELKPLEEVLNLAQSKNFHLRPRDLISNINVIVLELKGSE |
| | | TTFMCEYADETATIVEFLNRWITFVQSIISTLT |
| SEQ ID NO: 9 | ESE01.027 | MYRMQLLSCIALSLALVTNSAPTSSSTKKTQLQLEHLLLDL |
| | | QMILNGINNYKNPKLTEMLTQQFYMPKEATELKHLQCLEE |
| | | ELKPLEEVLNLAQSKNFHLRPRDLISDINVIVLELKGSETTF |
| | | MCEYADETATIVEFLNRWITFVQSIISTLT |
| SEQ ID NO: 10 | ESE01.028 | MYRMQLLSCIALSLALVTNSAPTSSSTKKTQLQLEHLLLDL |
| | | QMILNGINNYKNPKLTEMLTQQFYMPKEATELKHLQCLEE |
| | | ELKPLEEVLNLAQSKNFHLRPRDLISNINDIVLELKGSETTF |
| | | MCEYADETATIVEFLNRWITFVQSIISTLT |
| SEQ ID NO: 11 | ESE01.029 | MYRMQLLSCIALSLALVTNSAPTSSSTKKTQLQLEHLLLDL |
| | | QMILNGINNYKNPKLTEMLTQQFYMPKEATELKHLQCLEE |
| | | ELKPLEEVLNLAQSKNFHLRPRDLISNINAIVLELKGSETTF |
| | | MCEYADETATIVEFLNRWITFVQSIISTLT |
| SEQ ID NO: 12 | ESE01.030 | MYRMQLLSCIALSLALVTNSAPTSSSTKKTQLQLEHLLLDL |
| | | QMILNGINNYKNPKLTEMLTQQFYMPKEATELKHLQCLEE |
| | | ELKPLEEVLNLAQSKNFHLRPRDLISNINEIVLELKGSETTF |
| | | MCEYADETATIVEFLNRWITFVQSIISTLT |
| SEQ ID NO: 13 | ESE01.031 | MYRMQLLSCIALSLALVTNSAPTSSSTKKTQLQLEHLLLDL |
| | | QMILNGINNYKNPKLTEMLTQQFYMPKEATELKHLQCLEE |
| | | ELKPLEEVLNLAQSKNFHLRPRDLISNINVTVLELKGSETT |
| | | FMCEYADETATIVEFLNRWITFVQSIISTLT |
| SEQ ID NO: 14 | ESE01.032 | MYRMQLLSCIALSLALVTNSAPTSSSTKKTQLQLEHLLLDL |
| | | QMILNGINNYKNPKLTEMLTQQFYMPKEATELKHLQCLEE |
| | | ELKPLEEVLNLAQSKNFHLRPRDLISDINDIVLELKGSETTF |
| | | MCEYADETATIVEFLNRWITFVQSIISTLT |
| SEQ ID NO: 15 | ESE01.033 | MYRMQLLSCIALSLALVTNSAPTSSSTKKTQLQLEHLLLDL |
| | | QMILNGINNYKNPKLTEMLTQQFYMPKEATELKHLQCLEE |
| | | ELKPLEEVLNLAQSKNFHLRPRDLISDINAIVLELKGSETTF |
| | | MCEYADETATIVEFLNRWITFVQSIISTLT |
| SEQ ID NO: 16 | ESE01.034 | MYRMQLLSCIALSLALVTNSAPTSSSTKKTQLQLEHLLLDL |
| | | QMILNGINNYKNPKLTEMLTQQFYMPKEATELKHLQCLEE |
| | | ELKPLEEVLNLAQSKNFHLRPRDLISDINEIVLELKGSETTF |
| | | MCEYADETATIVEFLNRWITFVQSIISTLT |
| SEQ ID NO: 17 | ESE01.035 | MYRMQLLSCIALSLALVTNSAPTSSSTKKTQLQLEHLLLDL |
| | | QMILNGINNYKNPKLTEMLTQQFYMPKEATELKHLQCLEE |
| | | ELKPLEEVLNLAQSKNFHLRPRDLISNINDTVLELKGSETT |
| | | FMCEYADETATIVEFLNRWITFVQSIISTLT |
| SEQ ID NO: 18 | ESE01.036 | MYRMQLLSCIALSLALVTNSAPTSSSTKKTQLQLEHLLLDL |
| | | QMILNGINNYKNPKLTEMLTQQFYMPKEATELKHLQCLEE |
| | | ELKPLEEVLNLAQSKNFHLRPRDLISNINATVLELKGSETTF |
| | | MCEYADETATIVEFLNRWITFVQSIISTLT |
| SEQ ID NO: 19 | ESE01.037 | MYRMQLLSCIALSLALVTNSAPTSSSTKKTQLQLEHLLLDL |
| | | QMILNGINNYKNPKLTEMLTQQFYMPKEATELKHLQCLEE |
| | | ELKPLEEVLNLAQSKNFHLRPRDLISNINETVLELKGSETTF |
| | | MCEYADETATIVEFLNRWITFVQSIISTLT |
| SEQ ID NO: 20 | ESE01.038 | MYRMQLLSCIALSLALVTNSAPTSSSTKKTQLQLEHLLLDL |
| | | QMILNGINNYKNPKLTEMLTQQFYMPKEATELKHLQCLEE |
| | | ELKPLEEVLNLAQSKNFHLRPRDLISDINDTVLELKGSETT |
| | | FMCEYADETATIVEFLNRWITFVQSIISTLT |
| SEQ ID NO: 21 | ESE01.039 | MYRMQLLSCIALSLALVINSAPTSSSTKKTQLQLEHLLLDL |
| | | QMILNGINNYKNPKLTEMLTQQFYMPKEATELKHLQCLEE |
| | | ELKPLEEVLNLAQSKNFHLRPRDLISDINATVLELKGSETTF |
| | | MCEYADETATIVEFLNRWITFVQSIISTLT |
| SEQ ID NO: 22 | ESE01.040 | MYRMQLLSCIALSLALVTNSAPTSSSTKKTQLQLEHLLLDL |
| | | QMILNGINNYKNPKLTEMLTQQFYMPKEATELKHLQCLEE |
| | | ELKPLEEVLNLAQSKNFHLRPRDLISDINETVLELKGSETTF |
| | | MCEYADETATIVEFLNRWITFVQSIISTLT |
| SEQ ID NO: 23 | ESE01.042 | MYRMQLLSCIALSLALVTNSAPTSSSTKKTQLQLEHLLLDL |
| | | QMILNGINNYKNPKLTEMLTQQFYMPKKATELKHLQCLEE |
| | | ELKPLEEVLNLAQSKNFHLRPRDLISNINVIVLELKGSETTF |
| | | MCEYADETATIVEFLNRWITFVQSIISTLT |

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications, variations, improvements, and substantial equivalents.

1. An IL2 mutein, wherein the IL2 mutein with reduced or abolished binding to IL2Rα comprises an amino acid sequence comprising the amino acid substitution C125V or C125S as compared to wild type IL2 of SEQ ID NO:1.

2. The IL2 mutein of claim 1, wherein the IL2 mutein comprises one or more mutations selected from the group consisting of K8, 128, R38, F42, K43, K49, E61, E62, K64, E68, and L72, as compared to SEQ ID NO:2 or 3;
optionally,
the IL2 mutein comprises one or more amino acid substitutions selected from the group consisting of K8R, I28T, R38D/R38A/R38Q/R38T/R38E/R38N, F42E/F42R/F42A/F42H/F42Q, K43E/K43G/K43S/K43A/K43R/K43P/K43Q, K49E, E61R, E62T/E62G, K64M, E68G/E68V/E68S/E68K/E68A and L72S/L72K, as compared to the amino acid sequence set forth in SEQ ID NO: 2 or 3.

3. (canceled)

4. The IL2 mutein of claim 1, wherein the IL2 mutein comprises one or more amino acid substitutions selected from the group consisting of I28T, R38D/R38A/R38Q/R38T/R38E/R38N, K43E/K43G/K43S/K43A/K43R/K43P/K43Q, E61R and E68G/E68V/E68S/E68K/E68A; or
R38D/R38A/R38Q/R38T/R38E/R38N, F42E/F42R/F42A/F42H/F42Q and K43E/K43G/K43S/K43A/K43R/K43P/K43Q; or
R38D/R38A/R38Q/R38T/R38E/R38N, F42E/F42R/F42A/F42H/F42Q, K43E/K43G/K43S/K43A/K43R/K43P/K43Q, K49E; or
R38D/R38A/R38Q/R38T/R38E/R38N, F42E/F42R/F42A/F42H/F42Q, K43E/K43G/K43S/K43A/K43R/K43P/K43Q and E68G/E68V/E68S/E68K/E68A; or
F42E/F42R/F42A/F42H/F42Q and K43E/K43G/K43S/K43A/K43R/K43P/K43Q; or
K8R, R38D/R38A/R38Q/R38T/R38E/R38N, F42E/F42R/F42A/F42H/F42Q, K43E/K43G/K43S/K43A/K43R/K43P/K43Q, E62T/E62G, E68G/E68V/E68S/E68K/E68A and L72S/L72K; or
R38D/R38A/R38Q/R38T/R38E/R38N, E62T/E62G, E68G/E68V/E68S/E68K/E68A and L72S/L72K; or
R38D/R38A/R38Q/R38T/R38E/R38N, F42E/F42R/F42A/F42H/F42Q, K49E, K64M, K43E/K43G/K43S/K43A/K43R/K43P/K43Q and E68G/E68V/E68S/E68K/E68A,
as compared to the amino acid sequence set forth in SEQ ID NO: 2 or 3.

5. The IL2 mutein of claim 1, wherein the IL2 mutein comprises amino acid substitutions R38D, K43E/K43G and E61R;
R38A, F42E and K43G;
R38E, F42Q and K43Q;
R38A, F42E, K43G, E62T, E68G and L72S/L72K;
R38A, F42E, K43G, and E68G;
R38Q/R38T, F42R/F42A, K43S/K43A, and E68G/E68V;
R38A/R38T/R38E, E62T, E68S and L72K;
E62T/E62G, E68G and L72S;
R38E/R38N, K43R and I28T;
R38E/R38N, K43R, I28T and E68K;
F42H/F42Q and K43Q;
R38E, F42H/F42Q, K43Q and K49E;
R38D/R38E, F42Q, K43G/K43Q and K64M;
R38D/R38E, F42Q, K43G/K43Q and K49E; or
R38A/R38T, F42E/F42A, K43P, E62G, E68A and K8R,
as compared to the amino acid sequence set forth in SEQ ID NO: 2 or 3.

6. The IL2 mutein of claim 1, wherein the IL2 mutein comprises amino acid substitutions R38D, K43E and E61R;
R38A, F42E and K43G;
R38E, F42Q and K43Q;
R38A, F42E, K43G, E62T, E68G and L72S;
R38A, F42E, K43G and E68G;
R38Q, F42R, K43S and E68V;
R38T, F42A, K43A and E68G;
R38E, E62T, E68S and L72K;
E62T, E68G and L72S;
I28T, R38E and K43R;
I28T, R38N, K43R and E68K;
F42H and K43Q;
R38E, F42Q, K43Q and K49E;
R38D, F42Q, K43G and K64M; or
R38T, F42E, K43P, E62G, E68A and K8R;
as compared to the amino acid sequence set forth in SEQ ID NO: 2 or 3.

7. The IL2 mutein of claim 1, wherein the IL2 mutein comprises amino acid substitutions N88D, V91D/V91A/V91E and/or 192T as compared to the amino acid sequence set forth in SEQ ID NO: 2 or 3.

8. The IL2 mutein of claim 1, wherein the IL2 mutein comprises amino acid substitutions
R38E, F42Q, K43Q, K49E and N88D;
R38E, F42Q, K43Q, K49E and V91D/V91A/V91E;
R38E, F42Q, K43Q, K49E and 192T;
R38E, F42Q, K43Q, K49E, N88D and V91D/V91A/V91E;
R38E, F42Q, K43Q, K49E, V91D/V91A/V91E and I92T;
R38E, F42Q, K43Q, K49E, N88D, V91D/V91A/V91E and 192T;
I28T, R38E, K43R and N88D;
I28T, R38E, K43R and V91D/V91A/V91E;
I28T, R38E, K43R and 192T;
I28T, R38E, K43R, N88D and V91D/V91A/V91E;
I28T, R38E, K43R, V91D/V91A/V91E and 192T; or
I28T, R38E, K43R, N88D, V91D/V91A/V91E and 192T,
as compared to the amino acid sequence set forth in SEQ ID NO: 2 or 3.

9. The IL2 mutein of claim 1, wherein the IL2 mutein is at least 90%, 95%, 96%, 97%, 98%, 99% identical to the amino acid sequence set forth in any one of SEQ ID NOs: 7-23; or
the IL2 mutein is different from the amino acid sequence set forth in any one of SEQ ID NOs: 7-23 by no more than 10, 9, 8, 7, 6, 5, 4, 3, 2 or 1 amino acid.

10. (canceled)

11. The IL2 mutein of claim 1, wherein the IL2 mutein comprises the amino acid sequence set forth in any one of SEQ ID NOs: 7-23.

12. The IL2 mutein of claim 1, wherein the IL2 mutein is capable of reducing the affinity binding to IL2Rβγ heterodimer;
the IL2 mutein is capable of reducing the affinity binding to IL2Rαβγ heterotrimer by reduced or abolished binding to IL2Rα; or
the IL2 mutein is capable of reducing the immune response of regulatory T cells activated by IL2Rαβγ heterotrimer.

13. (canceled)

14. (canceled)

15. The IL2 mutein of claim 1, wherein the IL2 mutein binds to IL2Rβγ heterodimer with KD (affinity constant) of more than 0.1E-11M, more than 9.9E-11M, or more than 1E-10M;
the IL2 mutein binds to IL2Rα with KD (affinity constant) of more than 1.0E-12M; or
the IL2 mutein improves the thermostability with Tm value of not less than 50° C., not less than 55° C., not less than 60° C., or not less than 65° C.

16. (canceled)

17. (canceled)

18. An isolated polynucleotide encoding the IL2 mutein of claim 1.

19. An isolated vector comprising the polynucleotide of claim 18.

20. A host cell comprising the isolated vector of claim 19, wherein the host cell comprises HEK293 cells.

21. A fusion protein comprising the IL2 mutein of claim 1, wherein the IL2 mutein is fused to a Fc region, optionally via a linker.

22. A pharmaceutical composition comprising the IL2 mutein of claim 1, and a pharmaceutically acceptable carrier.

23. A method comprising:
using the IL2 mutein of claim 1 for the diagnosis, prevention or treatment of a disease, disorder or condition, optionally, the disease, disorder or condition comprises a tumor.

24. A method of diagnosing, preventing or treating a disease, disorder or condition in a subject in need thereof, comprising administrating to the subject a therapeutically effective amount of IL2 mutein of claim 1; optionally, the disease, disorder or condition comprises a tumor.

25. The method of claim 18, comprising:
enhancing the immune response to a IL2 related disease, disorder or condition in a subject in need thereof, or
reducing IL2 related adverse effects in antitumor immunotherapy in a subject in need thereof;
optionally, the subject is a mammal, optionally including a human.

26. (canceled)

27. (canceled)

28. (canceled)
